(19) United States
(12) Patent Application Publication
(10) Pub. No.: US 2008/0183231 A1
(43) Pub. Date: Jul. 31, 2008

(54) SYSTEMS, DEVICES AND METHODS TO ALTER AUTONOMIC TONE

(75) Inventors: Alok S. Sathaye, Boston, MA (US); Shantha Arcot-Krishnamurthy, Roseville, MN (US)

Correspondence Address: SCHWEGMAN, LUNDBERG & WOESSNER, P.A., P.O. BOX 2938, MINNEAPOLIS, MN, 55402, US

(73) Assignee: CARDIAC PACEMAKERS, INC., ST.PAUL, MN (US)

(21) Appl. No.: 11/668,631

(22) Filed: Jan. 30, 2007

(51) Int. Cl. A61N 1/362

(52) U.S. Cl. 607/9

(57) ABSTRACT

Various device embodiments include a pulse output circuit and a control circuit coupled to the pulse output circuit. The pulse output circuit is adapted to deliver electrical pacing pulses. The control circuit is adapted to receive an input signal indicative of a request to adjust autonomic tone, and is adapted to control the pulse output circuit in response to the request to deliver an overdrive pacing therapy to at least one cardiac region using the electrical pacing pulses.

### FIELD

This application relates generally to medical devices and, more particularly, to systems, devices and methods for providing cardiac pacing therapy.

### BACKGROUND

Atrial fibrillation (AF) is the most common cardiac arrhythmia. AF affects millions of people. Currently, drug based therapy is used to either prevent induction of AF (rhythm control) or lessen the systemic effects of the disease by controlling the ventricular rate (rate control). Device-based therapies, such as atrial antitachycardia pacing/defibrillation and dynamic atrial overdrive pacing, have been ineffective in treating the disease. There exists a need to prevent AF by a non-drug type therapy

### SUMMARY

Various device embodiments include a pulse output circuit and a control circuit coupled to the pulse output circuit. The pulse output circuit is adapted to deliver electrical pacing pulses. The control circuit is adapted to receive an input signal indicative of a request to adjust autonomic tone, and is adapted to control the pulse output circuit in response to the request to deliver an overdrive pacing therapy to at least one cardiac region using the electrical pacing pulses.

Various system embodiment include means for receiving a request to adjust autonomic tone, and means for delivering an overdrive pacing therapy to at least one cardiac region in response to the request to adjust autonomic tone. According to various method embodiments, a request to adjust autonomic tone is received, and an overdrive pacing therapy is delivered to at least one cardiac region in response to the request to adjust autonomic tone.

This Summary is an overview of some of the teachings of the present application and not intended to be an exclusive or exhaustive treatment of the present subject matter. Further details about the present subject matter are found in the detailed description and appended claims. Other aspects will be apparent to persons skilled in the art upon reading and understanding the following detailed description and viewing the drawings that form a part thereof, each of which are not to be taken in a limiting sense. The scope of the present invention is defined by the appended claims and their equivalents.

### BRIEF DESCRIPTION OF DRAWINGS

FIG. 1 is a graph illustrating various pacing methods and their effects on autonomic tone as a function of pacing rate.

FIG. 2 illustrates a device embodiment to adjust autonomic tone.

FIG. 3 illustrates a method for delivering pacing therapy to adjust autonomic tone in a open-loop format according to various embodiments.

FIG. 4 illustrates a pacing device according to various embodiments.

FIG. 5 illustrates a device embodiment, with illustrated examples of inputs affecting delivery of autonomic pacing therapies.

FIG. 6 illustrates a method for delivering pacing therapy to adjust autonomic tone according to various embodiments.

FIG. 7 illustrates an implantable medical device (IMD) having an autonomic pacing component and a cardiac rhythm management (CRM) component according to various embodiments.

FIG. 8 illustrates a device diagram of a microprocessor-based pacing device according to various embodiments.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various embodiments employ atrial and ventricular overdrive pacing to influence autonomic tone to prevent atrial fibrillation (AF) or other parasympathetically-mediated pathologies including vaso-vagal syncope. In various embodiments, a device monitors autonomic balance indicators (ABIs) such as heart rate and heart rate variability, and delivers intermittent pacing in the atrium or ventricle depending upon the autonomic balance of the patient. In various embodiments, the device delivers atrial pacing to influence the Autonomic Nervous System (ANS) to increase heart rate when the heart rate is depressed. In various embodiments, when HRV indicates a highly parasympathetic autonomic balance, the device delivers ventricular overdrive pacing to increase sympathetic tone. In various embodiments, where measured autonomic balance is highly sympathetic, the device delivers intermittent ventricular overdrive pacing therapy to elicit an increased parasympathetic tone.

AF is the most common cardiac arrhythmia. AF affects millions of people. Increased parasympathetic tone is a common induction mechanism for AF. Preventing AF and other parasympathetically mediated pathologies can be accomplished by encouraging the ANS to respond to artificial stimuli in a manner to counteract the anticipated undesirable cardiac event. The ANS uses various baroreceptors and chemoreceptors to maintain hemodynamic stability in the body. The body is hemodynamically balanced when the cardiac output of the heart matches the body's metabolic demands. The ANS attempts to maintain the hemodynamic balance in response to a perturbed hemodynamic system. Fear, exercise and anger are examples of perturbations that increase the sympathetic activity or tone of the ANS. Increased sympathetic tone tends to increase heart rate and blood pressure as well increase blood flow to skeletal muscles while decreasing blood flow to the digestive tract. Likewise, sleep and inactivity are examples of events that lead to increased parasympathetic activity or tone of the ANS. Increased parasympathetic tone tends to lower heart rate and blood pressure as well as increase blood flow and activity of organs involved with rest and recovery efforts of the body such as digestion. The apparatus and methods described herein use the reactive nature of the ANS in response to stimuli to balance the autonomic tone of the ANS in a predictive manner so as to prevent and/or remedy various heart ailments.

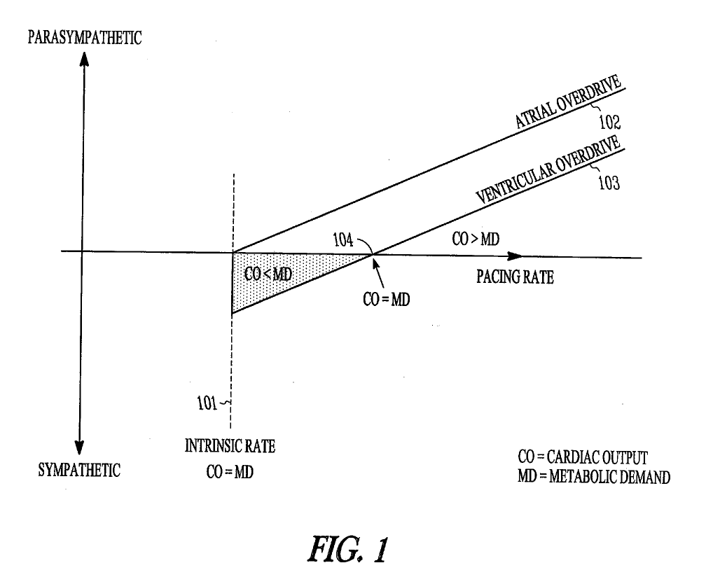

FIG. 1 illustrates anticipated autonomic response to various pacing scenarios as the pacing rate is increased above a patient's intrinsic pacing rate 101. A patient's intrinsic rate varies and depends on many circumstances. Generally, a patient's intrinsic rate increases when the patient is active, such as when exercising, and decreases when the patient is inactive, such as when sleeping. Pacing therapy can be delivered in many forms. FIG. 1 illustrates the effect of overdrive pacing as a function of pacing rate. The graph illustrates the relationships of autonomic tone and pacing as a linear relationship for the purposes of simplicity and clarity. The effect of overdrive pacing is likely non-linear and patient-specific. The graph shows a line for both atrial overdrive pacing 102 and ventricular overdrive pacing 103. The atrial overdrive pacing graph line shows that pacing an atrial chamber at rates above the patient's intrinsic pacing rate will result in a parasympathetic response. The atrial overdrive pacing therapy increases the heart rate to a rate greater than the intrinsic rate, resulting in cardiac output (CO) being higher then the metabolic demand (MD) of the patient. The ANS responds to the higher CO by increasing the parasympathetic tone in an effort to reduce CO.

Ventricular overdrive pacing can also affect CO, causing the ANS to adjust autonomic tone to restore hemodynamic balance. Ventricular pacing often causes the ventricle to operate in an inefficient manner such that cardiac output does not meet metabolic demand. Thus, lower ventricular overdrive pacing rates lower CO, eliciting a sympathetic response to increase the intrinsic rate of the heart to accommodate the induced inefficiency. Increased ventricular pacing allows CO to meet the patient's metabolic demands (MD), as illustrated at 104. Further increases in ventricular pacing increases CO, eliciting a parasympathetic response in an effort to retard cardiac output.

FIG. 2 illustrates a device embodiment to adjust autonomic tone. The illustrated device 210 includes a therapy control circuit 212, a pacing circuit 213 and pacing electrode(s) 214 to deliver the pacing stimulation pulses to the heart. The control circuit 212 responds to an autonomic balance therapy request 211 to control the pacing circuit to deliver overdrive pacing to the heart. The circuitry is capable of being implemented using hardware, software, and combinations of hardware and software. The figure also illustrates an enable signal 215, which represents the ability of the patient to override the autonomic pacing therapy. The enable functionality may be implemented in many forms. According to various embodiments where the device 210 is an implantable medical device, the patient can trigger the enable circuit remotely when the therapy conflicts with the patients activity or the therapy results in discomfort. The enable signal may also be based on a schedule. For example, if a patient is scheduled to receive intermittent overdrive pacing throughout the day, the enable signal can be programmed to only allow therapy during times corresponding to normal waking hours so as not to disturb a patient's sleep. Other physiological factors, such as metabolic demand, can be used to determine when to enable the therapy. Either the control circuit 212 or the pacing circuit 213 or both can be adapted to receive and respond to the enable signal 215 to allow overdrive pacing therapy.

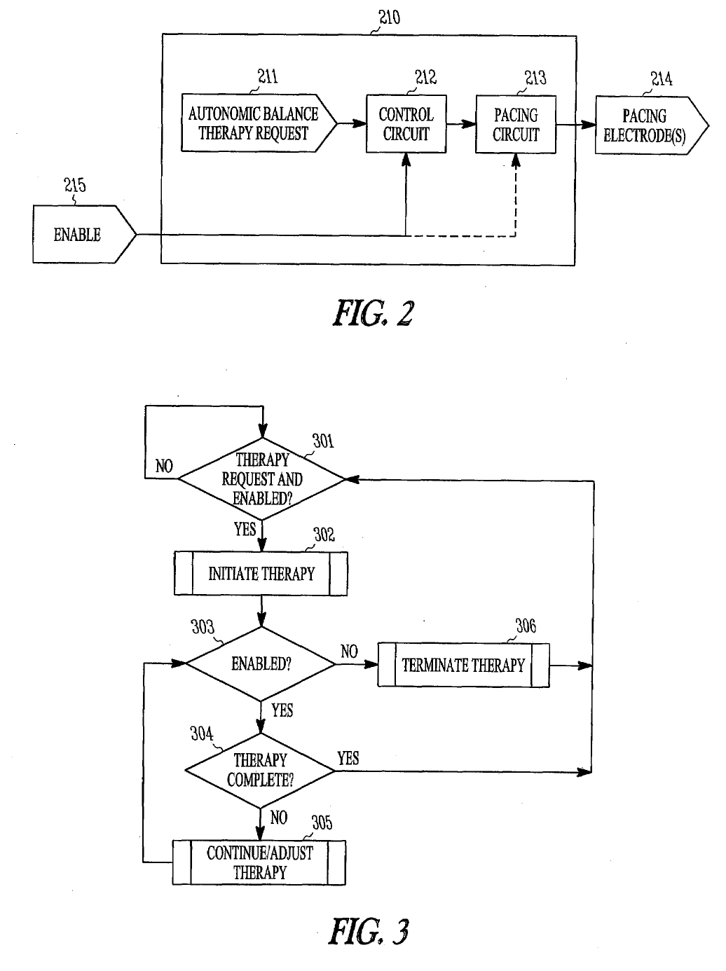

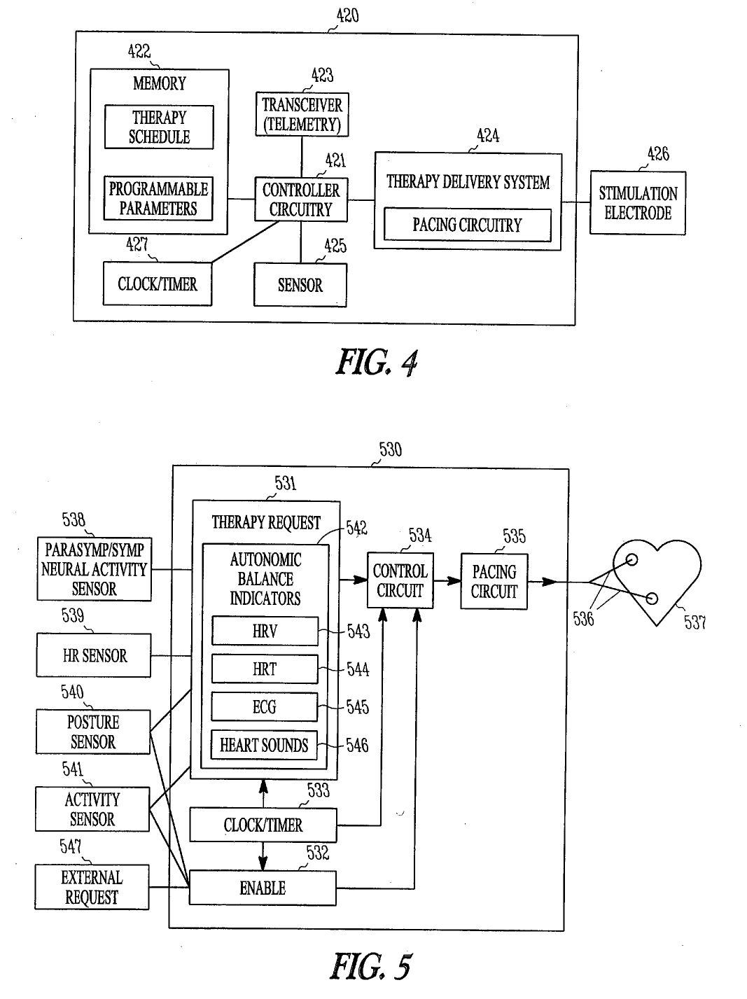

FIG. 3 illustrates a method for delivering overdrive pacing therapy to adjust autonomic tone in an open-loop format according to various embodiments. The illustrated process monitors a request for overdrive pacing therapy as well as the therapy enable signal at 301. When a therapy is requested and enabled, the therapy is initiated at 302. The device continues to monitor the enable signal 303 and for the completion of the therapy 304 as the therapy initiated at 302 continues at 305. When the therapy is completed, as illustrated at 304, the process returns to monitor for the initiation of the next therapy request. If, during the delivery of therapy 305, the therapy is no longer enabled, as identified at 303, the therapy is terminated at 306 and the process returns to 301 to monitor the next enabled therapy request.

FIG. 4 illustrates an implantable medical device (IMD) 420, according to various embodiments. The illustrated IMD 420 provides pacing signals for delivery to predetermined targets within the heart to provide a desired therapy. The illustrated device includes controller circuitry 421 and memory 422. The controller circuitry 421 is capable of being implemented using hardware, software, and combinations of hardware and software. For example, according to various embodiments, the controller circuitry 421 includes a processor to perform instructions embedded in the memory 422 to perform functions associated with the pacing therapy. The illustrated device further includes a transceiver 423 and associated circuitry for use to communicate with a programmer or another external or internal device. Various embodiments have wireless communication capabilities. For example, some transceiver embodiments use a telemetry coil to wirelessly communicate with a programmer or another external or internal device. Such a device can be an enabling device to enable and disable autonomic overdrive pacing therapy according to the patient's desire.

The illustrated device further includes a therapy delivery system 424, including pacing circuitry. The pacing circuitry is used to apply electrical stimulation pulses to desired cardiac targets using one or more pacing electrodes 426 positioned at predetermined location(s). Various embodiments of the device also include sensor circuitry 425. According to some embodiments, one or more leads are able to be connected to the sensor circuitry 425 and pacing circuitry 424. Some embodiments use wireless connections between the sensor(s) and sensor circuitry 425. The pacing circuitry is used to apply electrical stimulation pulses to desired cardiac targets, such as through one or more pacing electrodes 426 positioned at predetermined location(s).

According to various embodiments using pacing stimulation, the stimulation circuitry 424 is adapted to set or adjust any one or any combination of pacing features. Examples of pacing features include, but are not limited to, the amplitude, frequency and polarity of the pacing signal. The controller 421 can be programmed to control the pacing stimulation delivered by the pacing circuitry 424 according to pacing instructions, such as a pacing schedule, stored in the memory 422. Pacing stimulation can be delivered in a step function. The pacing stimulation can be delivered with an increasing ramp function at the beginning of the stimulation, a decreasing ramp function at the end of the stimulation, or both an increasing ramp function and decreasing ramp function. Changing the rate according to a step function is believed to be more effective in eliciting an autonomic response than changing the pacing rate gradually according to a ramp function, but it is believed that a patient is able to tolerate pacing rate adjustments according to a ramp function easier than according to a step function.

In various embodiments, the sensor circuitry 425 is adapted to detect physiological responses. Examples of physiological responses include blood pressure, cardiac activity such as heart rate, and respiration such as tidal volume and minute ventilation. The controller circuitry can control the therapy using a therapy schedule in memory 422, and/or can compare a target range (or ranges) of the sensed physiological response(s) stored in the memory 422 to the sensed physiological response(s) to appropriately adjust or trigger autonomic pacing therapy. The controller 421 compares the response to a target range stored in memory, and controls the overdrive pacing therapy based on the comparison in an attempt to keep the response within the target range. The target range can be programmable.

The illustrated device includes a clock or timer 427 which can be used to execute the programmable pacing schedule. For example, a clinician can program a daily schedule of therapy based on the time of day. A pacing session can begin at a first programmed time, and can end at a second programmed time. According to various embodiments, the schedule refers to the time intervals or the period when the pacing therapy is delivered. A schedule can be defined by a start time and an end time, a start time and a duration, a start time and a terminating event, an initiating trigger and an end time, an initiating trigger and a duration or an initiating trigger and a terminating event. Various schedules deliver therapy periodically. According to various examples, a device can be programmed with a therapy schedule to deliver therapy from midnight to 2 AM every day, or to deliver therapy for one hour every six hours, or to deliver therapy for two hours per day, or according to a more complicated timetable. Various device embodiments apply the therapy according to the programmed schedule contingent on enabling conditions, such as patient rest or sleep, low heart rate levels, and the like. Various embodiments initiate and/or terminate a pacing session based on a signal triggered by a patient or clinician. Various embodiments use sensed data to enable and/or disable a pacing session.

FIG. 5 illustrates a device embodiment to deliver overdrive pacing with illustrated examples of inputs that can effect delivery of autonomic pacing therapies. The illustrated device 530 includes inputs connected to a therapy request module 531 and/or an enable module 532. The enable module 532 may respond to an external request 547, such as may be provided by an external programmer or magnet, for example. The therapy request module 531, the enable module 532 and a clock timer module 533 are connected to a control circuit 534. The control circuit 534 controls the pacing therapy and sends pacing command signals to a pacing circuit 535. The illustrated pacing circuit 535 delivers pacing pulses according to the pacing commands to the heart 537 through leads 536 using electrodes. In various embodiments, some of the inputs connect to the therapy request module 531 and the enable module 532 to initiate overdrive pacing therapy and also provide feedback used to control ongoing therapy. Various embodiments include physiological sensors such as a parasympathetic and/or a sympathetic neural activity sensor 538, a heart rate (HR) sensor 539, a posture sensor 540 and an activity sensor 541. Various embodiments can also include a blood pressure sensor and a respiration sensor to sense tidal volume, minute ventilation and/or a cardiovascular respiration relationship (CVRR). CVRR is the ratio of cardiac cycles to respiratory cycles. The sensors are monitored by the therapy request module 531 to determine when, and what type of overdrive pacing therapy is indicated. Within the therapy request module 531, the various physiological inputs can be used to derive additional indicators of autonomic balance 542. Such derived autonomic balance indicators (ABIs) 542 include heart rate variability (HRV) 543, heart rate turbulence (HRT) 544, electrocardiogram (ECG) data 545, which may use the pacing electrodes 536, and heart sounds 546. ABIs can also include indicator(s) of parasympathetic nerve activity, sympathetic nerve activity, or both parasympathetic and sympathetic nerve activity, and can also include CVRR. The value of the derived ABIs can be compared to preprogrammed threshold values, or trends, to trigger overdrive pacing therapy to prevent anticipated cardiac events, including parasympathetically mediated atrial fibrillation (AF) or an impending syncope.

The illustrated embodiment of FIG. 5 also includes a clock timer circuit 533. The clock/timer 533 facilitates the functionality of the therapy request module 531, the enable module 532 and the control circuit 534. With respect to the therapy request module 531, the clock/timer module 533 assists in triggering scheduled, overdrive pacing therapy. The clock/timer module 533 also assists the functionality of the ABIs 542 in providing a time reference from which calculations and recordings can be based. With respect to the enable module 532, the clock/timer assists in allowing therapy to be enabled. The enable module can follow preprogrammed rules to determine whether overdrive pacing therapy is enabled or disabled. A determination can rely on the time of day, the duration and conclusion of previous therapy, as well as, the posture of the patient and the patient's current and/or recorded activity level. With respect to the control circuit 534, the clock/timer module 533 assists, among other things, in processing and timing the actual therapy.

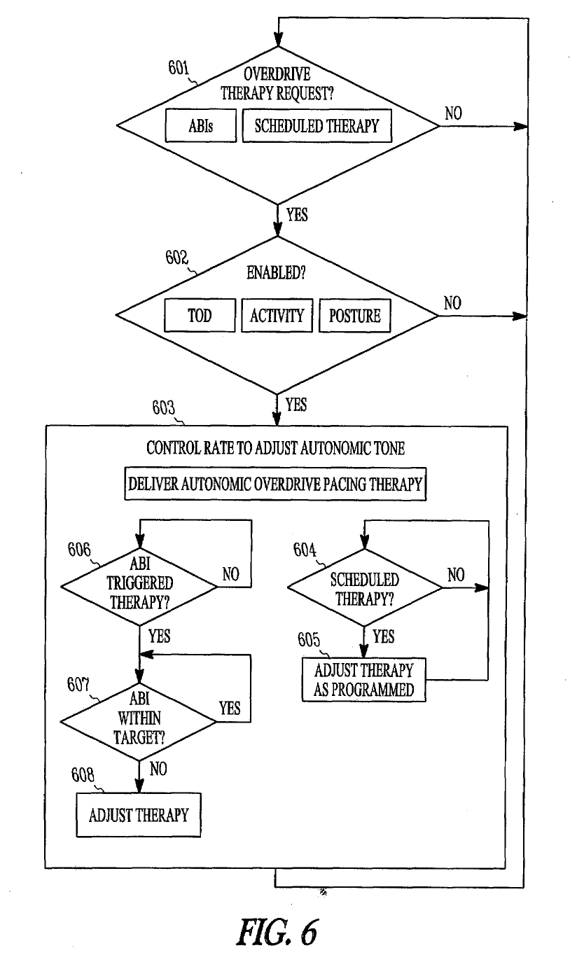

FIG. 6 illustrates a method for delivering therapy to adjust a patient's autonomic tone, according to various embodiments. At 601, it is determined whether overdrive pacing therapy is desired. Various embodiments use preprogrammed pacing schedules and an internal clock or timer to determine if overdrive pacing is desired. Additionally, various embodiments will monitor physiological sensors as well as Autonomic Balance Indicators derived from the physiological sensors, clocks and/or timers to determine when overdrive pacing may be desirable to either prevent parasympathetically mediated arrhythmias, or other cardiac anomalies, or remedy a detected arrhythmia or other anomaly, such as an impending syncope. When it is determined that overdrive pacing is desired, the process proceeds to 602 to determine whether overdrive pacing is enabled. Such a determination may involve heart rate measurements, activity measurements, posture measurements, correlation with a timer or clock and various combinations thereof.

When it is determined that overdrive pacing is enabled, the process proceeds to 603 to deliver overdrive pacing to adjust the autonomic tone of the patient. If the therapy request originated from a scheduled preprogrammed basis 604, the process will continue to monitor other potential therapy triggers while delivering the therapy according to the preprogrammed regiment and schedule 605. If the therapy request originated from a detected anomaly or triggering condition based on physiological indicators or ABIs 606, the process will deliver the therapy until the physiological indicators or ABIs are within an acceptable range 607 to terminate or adjust the therapy according to preprogrammed rules 608. Various embodiments deliver a scheduled therapy and use sensed ABIs to titrate the therapy.

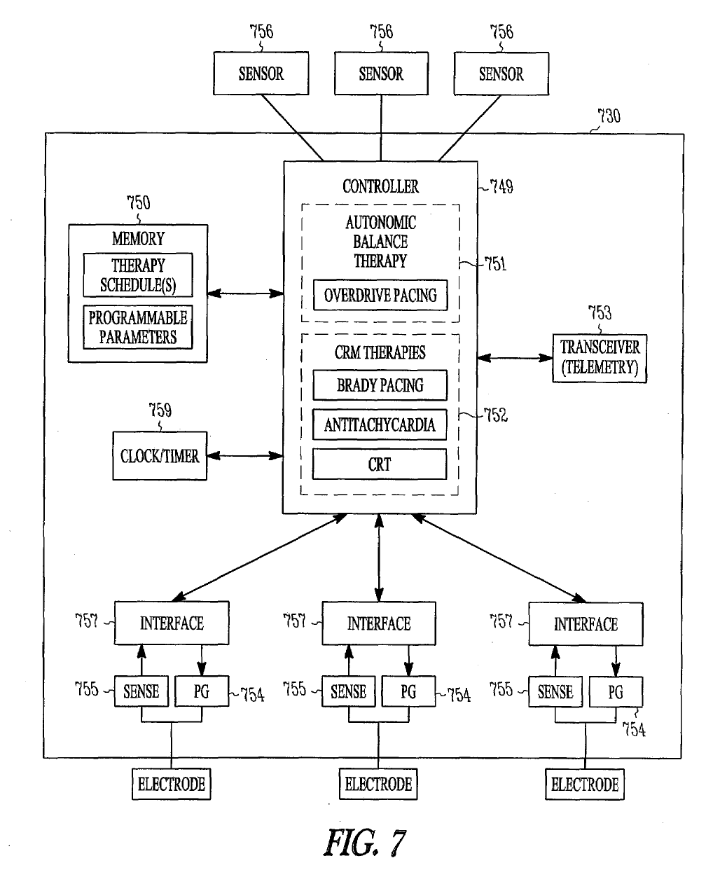

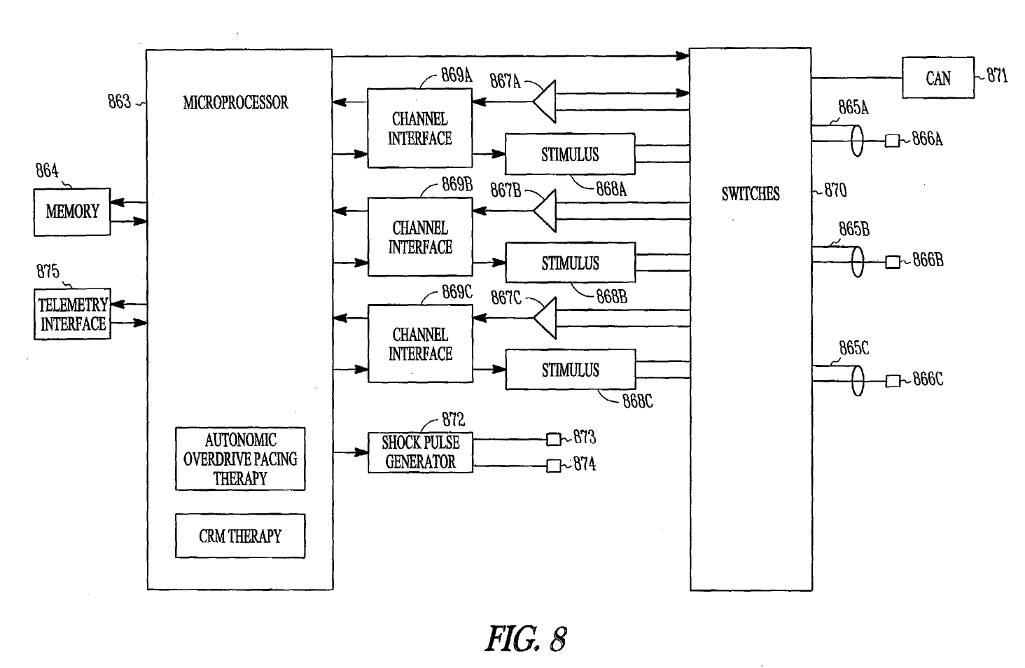

FIG. 7 illustrates an implantable medical device (IMD) 730 having an autonomic balance therapy component 751 and a cardiac rhythm management (CRM) component 752 according to various embodiments of the present subject matter. The illustrated device includes a controller 749 and memory 750. According to various embodiments, the controller includes hardware, software, or a combination of hardware and software to perform the overdrive pacing and CRM functions. For example, the programmed therapy applications discussed in this disclosure are capable of being stored as computer-readable instructions embodied in memory and executed by a processor. For example, therapy schedule(s) and programmable parameters can be stored in memory. According to various embodiments, the controller includes a processor to execute instructions embedded in memory to perform the overdrive pacing and CRM functions. The illustrated autonomic balance therapy 751 may include therapies to elicit a parasympathetic response using either atrial overdrive pacing or ventricular overdrive pacing at pacing rates higher than the intrinsic rate and higher than a rate that would elicit a sympathetic response. The illustrated autonomic balance therapy 751 may include therapies to elicit a sympathetic response via ventricular overdrive pacing at pacing rates higher then the patients intrinsic rate but lower than a rate that would induce a parasympathetic response from the autonomic nervous system. Ventricular pacing often causes the ventricle to operate in an inefficient manner such that cardiac output does not meet metabolic demand. Thus lower ventricular overdrive pacing rates lower CO, eliciting a sympathetic response to increase the intrinsic rate of the heart to accommodate the induced inefficiency. Increased ventricular pacing allows CO to meet the patient's metabolic demands (MD). Further increases in ventricular pacing increases CO, eliciting a parasympathetic response in an effort to retard cardiac output. Various embodiments include CRM therapies 752, such as bradycardia pacing, anti-tachycardia therapies such as ATP, defibrillation and cardioversion, and cardiac resynchronization therapy (CRT). The illustrated device further includes a transceiver 753 and associated circuitry for use to communicate with a programmer or another external or internal device. Various embodiments include a telemetry coil.

The CRM and autonomic balance therapy sections, 751 and 752, include components, under the control of the controller, to stimulate a heart and/or sense cardiac signals using one or more electrodes. The illustrated CRM and autonomic balance therapy sections include a pulse generator 754 for use to provide an electrical signal through an electrode to stimulate a heart, and further includes sense circuitry 755 to detect and process sensed cardiac signals. An interface 757 is generally illustrated for use to communicate between the controller 749 and the pulse generator 754 and sense circuitry 755. Three electrodes are illustrated as an example for use to provide CRM or autonomic balance therapy. However, the present subject matter is not limited to a particular number of electrode sites. Each electrode may include its own pulse generator and sense circuitry. However, the present subject matter is not so limited. The pulse generating and sensing functions can be multiplexed to function with multiple electrodes.

The illustrated device further includes a clock/timer 759, which can be used to deliver the programmed therapy according to a programmed pacing protocol and/or schedule. In various embodiments, the device includes various sensors. The sensor inputs 756 may be connected to one or more physiological sensors. A heart rate monitor, blood pressure monitor, posture sensor, activity sensor and ECG sensors are examples of sensors. Physiological sensors can be used to determine when overdrive pacing therapy is appropriate. Physiological sensors can also be used to assist in assessing the autonomic tone. The autonomic tone can be correlated to other data to trigger overdrive pacing therapy to prevent parasympathetically mediated arrhythmias, or other cardiac anomalies, or remedy a detected arrhythmia or other anomaly, such as an impending syncope. The assessed autonomic tone can also be used as therapy is delivered, such that adjustments can be made to ensure the therapy is more efficacious.

For patients with a history of AF or parasympathetically mediated pathologies, the physiological sensors 756 as well as the clock/timer 759 can assist in detecting periods of high risk for anticipated cardiac anomalies and trigger preventative pacing therapies or intensify monitoring to determine whether intervention pacing therapy is required.

FIG. 8 shows a system diagram of an embodiment of a microprocessor-based implantable device, according to various embodiments. The controller of the device is a microprocessor 863 which communicates with a memory 864 via a bidirectional data bus. The controller could be implemented by other types of logic circuitry (e.g., discrete components or programmable logic arrays) using a state machine type of design. As used herein, the term “circuitry” should be taken to refer to either discrete logic circuitry or to the programming of a microprocessor. Shown in the figure are three examples of sensing and pacing channels designated “A” through “C” comprising bipolar leads with ring electrodes 865A-C and tip electrodes 866A-C, sensing amplifiers 867A-C, pulse generators 868A-C, and channel interfaces 869A-C. Each channel thus includes a pacing channel made up of the pulse generator connected to the electrode and a sensing channel made up of the sense amplifier connected to the electrode. The channel interfaces 869A-C communicate bidirectionally with the microprocessor 863, and each interface may include analog-to-digital converters for digitizing sensing signal inputs from the sensing amplifiers and registers that can be written to by the microprocessor in order to output pacing pulses, change the pacing pulse amplitude, and adjust the gain and threshold values for the sensing amplifiers. The sensing circuitry of the pacemaker detects a chamber sense, either an atrial sense or ventricular sense, when an electrogram signal (i.e., a voltage sensed by an electrode representing cardiac electrical activity) generated by a particular channel exceeds a specified detection threshold. Pacing algorithms used in particular pacing modes employ such senses to trigger or inhibit pacing. The intrinsic atrial and/or ventricular rates can be measured by measuring the time intervals between atrial and ventricular senses, respectively, and used to detect atrial and ventricular tachyarrhythmias.

The electrodes of each bipolar lead are connected via conductors within the lead to a switching network 870 controlled by the microprocessor. The switching network is used to switch the electrodes to the input of a sense amplifier in order to detect intrinsic cardiac activity and to the output of a pulse generator in order to deliver a pacing pulse. The switching network also enables the device to sense or pace either in a bipolar mode using both the ring and tip electrodes of a lead or in a unipolar mode using only one of the electrodes of the lead with the device housing (CAN) 871 or an electrode on another lead serving as a ground electrode. A shock pulse generator 872 is also interfaced to the controller for delivering a defibrillation shock via a pair of shock electrodes 873 and 874 to the atria or ventricles upon detection of a shockable tachyarrhythmia.

The figure illustrates a telemetry interface 875 connected to the microprocessor, which can be used to communicate with an external device. The illustrated microprocessor 863 is capable of performing autonomic overdrive pacing therapy routines and myocardial (CRM) stimulation routines. Examples of autonomic overdrive pacing therapy routines include atrial overdrive pacing and ventricular overdrive pacing to elicit a parasympathetic tone from the autonomic nervous system. Additionally, ventricular overdrive pacing therapy can be applied in a manner to elicit a sympathetic tone from the autonomic nervous system. Ventricular overdrive pacing therapy to elicit a sympathetic response can be accomplished by pacing the ventricle at a rate greater than the patient's intrinsic rate but less than a rate that will elicit a parasympathetic response. Examples of myocardial therapy routines include bradycardia pacing therapies, anti-tachycardia shock therapies such as cardioversion or defibrillation therapies, anti-tachycardia pacing therapies (ATP), and cardiac resynchronization therapies (CRT).

One of ordinary skill in the art will understand that, the modules and other circuitry shown and described herein can be implemented using software, hardware, and combinations of software and hardware. As such, the terms module and circuit are intended to encompass software implementations, hardware implementations, and software and hardware implementations.

The methods illustrated in this disclosure are not intended to be exclusive of other methods within the scope of the present subject matter. Those of ordinary skill in the art will understand, upon reading and comprehending this disclosure, other methods within the scope of the present subject matter. The above-identified embodiments, and portions of the illustrated embodiments, are not necessarily mutually exclusive. These embodiments, or portions thereof, can be combined. In various embodiments, the methods are implemented using a computer data signal embodied in a carrier wave or propagated signal, that represents a sequence of instructions which, when executed by a processor cause the processor to perform the respective method. In various embodiments, the methods are implemented as a set of instructions contained on a computer-accessible medium capable of directing a processor to perform the respective method. In various embodiments, the medium is a magnetic medium, an electronic medium, or an optical medium.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A device, comprising:
a pulse output circuit adapted to deliver electrical pacing pulses; and
a control circuit coupled to the pulse output circuit, the control circuit adapted to receive an input signal indicative of a request to adjust autonomic tone and adapted to control the pulse output circuit in response to the request to deliver an overdrive pacing therapy to at least one cardiac region using the electrical pacing pulses.

2. The device of claim 1, wherein:
the input signal is indicative of a request to increase parasympathetic tone;
the pulse output circuit is adapted to deliver atrial overdrive pacing to an atrial region; and
the control circuit is adapted to control the pulse output circuit in response to the input signal to elicit a parasympathetic response using atrial overdrive pacing.

3. The device of claim 1, wherein:
the input signal is indicative of a request to increase sympathetic tone;
the pulse output circuit is adapted to deliver ventricular overdrive pacing to a ventricular region; and
the control circuit is adapted to control the pulse output circuit in response to the input signal to elicit a sympathetic response using ventricular overdrive pacing.

4. The device of claim 1, wherein:
the input signal is indicative of a request to increase parasympathetic tone;
the pulse output circuit is adapted to deliver ventricular overdrive pacing; and
the control circuit is adapted to control the pulse output circuit in response to the input signal to elicit a parasympathetic response using ventricular overdrive pacing.

5. The device of claim 1, wherein:
the pulse output circuit is adapted to deliver ventricular overdrive pacing at a first rate within a first range of overdrive pacing rates and at a second rate within a second range of overdrive pacing rates, the first range of overdrive pacing rates being lower than the second range of overdrive pacing rates; and
the control circuit is adapted to receive a first input signal indicative of a request to increase parasympathetic tone at a first time and a second input signal indicative of a request to increase sympathetic tone at a second time distinct from the first time, and is
further adapted to control the pulse output circuit in response to the second input signal to elicit a sympathetic response using ventricular overdrive pacing at the first rate, and to control the pulse output circuit in response to the first input signal to elicit a parasympathetic response using ventricular overdrive pacing at the second rate.

6. The device of claim 1, wherein:
the pulse output circuit is adapted to deliver atrial overdrive pacing and to deliver ventricular overdrive pacing; and
the control circuit is adapted to receive a first input signal indicative of a request to increase parasympathetic tone at a first time and a second input signal indicative of a request to increase sympathetic tone at a second time distinct from the first time, and is further adapted to control the pulse output circuit in response to the first input signal to elicit a parasympathetic response using atrial overdrive pacing, and to control the pulse output circuit in response to the second input signal to elicit a sympathetic response using ventricular overdrive pacing.

7. The device of claim 1, wherein the control circuit or the pulse output circuit is adapted to receive a therapy enable signal and to respond to the therapy enable signal by allowing the overdrive pacing therapy in response to the request to adjust autonomic tone.

8. The device of claim 7, wherein the device is implantable and the therapy enable signal is an externally-generated signal, the device further including a communication circuit to receive the externally-generated signal and provide the therapy enable signal to the control circuit or the pulse output circuit.

9. The device of claim 1, wherein the input signal indicative of a request to adjust autonomic tone includes a signal based on an autonomic balance indicator (ABI).

10. The device of claim 9, wherein, the autonomic balance indicator includes a detected Heart Rate Variability (HRV), detected Heart Rate Turbulence (HRT), a cardiovascular respiration relationship (CVRR), or an indicator of parasympathetic and sympathetic nerve activity.

11. The device of claim 1, wherein the input signal indicative of a request to adjust autonomic tone includes a signal from a physiological sensor.

12. The device of claim 11, wherein the physiological sensor indicates heart rate.

13. The device of claim 11, wherein the physiological sensor indicates blood pressure.

14. The device of claim 1, wherein the control circuitry is adapted to ramp up a pacing rate in preparation to deliver the overdrive pacing therapy, or to ramp down the pacing rate after delivering the overdrive pacing therapy, or to ramp up the pacing rate in preparation to deliver the overdrive pacing therapy and to ramp down the pacing rate after delivering the overdrive pacing therapy.

15. A method, comprising:
receiving a request to adjust autonomic tone; and
delivering an overdrive pacing therapy to at least one cardiac region in response to the request to adjust autonomic tone.

16. The method of claim 15, wherein:
receiving a request to adjust autonomic tone includes receiving a signal to increase parasympathetic tone; and
delivering an overdrive pacing therapy to at least one cardiac region includes delivering overdrive pacing therapy to an atrial region to elicit a parasympathetic response.

17. The method of claim 15, wherein:
receiving a request to adjust autonomic tone includes receiving a signal to increase sympathetic tone; and
delivering an overdrive pacing therapy to at least one cardiac region includes delivering overdrive pacing therapy to a ventricular region to elicit a sympathetic response.

18. The method of claim 15, wherein:
receiving a request to adjust autonomic tone includes receiving a signal to increase parasympathetic tone; and
delivering an overdrive pacing therapy to at least one cardiac region includes delivering overdrive pacing therapy to a ventricular region to elicit a parasympathetic response.

19. The method of claim 15, wherein:
receiving a request to adjust autonomic tone includes:
receiving a first signal to increase parasympathetic tone at a first time; and
receiving a second signal to increase sympathetic tone at a second time distinct from the first time; and
delivering an overdrive pacing therapy to at least one cardiac region in response to the request to adjust autonomic tone includes:
delivering overdrive pacing therapy to a ventricular region at a first pacing rate to elicit a parasympathetic response in response to the first signal; and
delivering overdrive pacing therapy to a ventricular region at a second pacing rate to elicit a sympathetic response in response to the second signal, the second pacing rate being less than the first pacing rate.

20. The method of claim 15, wherein:
receiving a request to adjust autonomic tone includes:
receiving a first signal to increase parasympathetic tone at a first time; and
receiving a second signal to increase sympathetic tone at a second time distinct from the first time; and
delivering an overdrive pacing therapy to at least one cardiac region in response to the request to adjust autonomic tone includes:
delivering overdrive pacing therapy to an atrial region to elicit a parasympathetic response in response to the first signal; and
delivering overdrive pacing therapy to a ventricular region to elicit a sympathetic response in response to the second signal.

21. The method of claim 15, further comprising:
monitoring an autonomic balance indicator; and
generating the request to adjust autonomic tone when a value for the autonomic balance indicator is outside an autonomic tone threshold limit.

22. The method of claim 21, wherein monitoring at least one autonomic indicator includes monitoring heart rate variability (HRV).

23. The method of claim 15, further comprising:
monitoring a physiological input;
generating the request to adjust autonomic tone when a value for the physiological input is outside an autonomic tone threshold limit.

24. The method of claim 23, wherein monitoring a physiological input includes monitoring heart rate.

25. The method of claim 23, wherein monitoring a physiological input includes monitoring blood pressure.

26. A system, comprising:
means for receiving a request to adjust autonomic tone; and
means for delivering an overdrive pacing therapy to at least one cardiac region in response to the request to adjust autonomic tone.

27. The system of claim 26, wherein:
means for receiving a request to adjust autonomic tone includes means for receiving a signal to increase parasympathetic tone; and
means for delivering an overdrive pacing therapy to at least one cardiac region in response to the request to adjust autonomic tone includes means for delivering overdrive pacing therapy to an atrial region to elicit a parasympathetic response.

28. The system of claim 26, wherein:
means for receiving a request to adjust autonomic tone includes means for receiving a signal to increase sympathetic tone; and
means for delivering an overdrive pacing therapy to at least one cardiac region in response to the request to adjust autonomic tone includes means for delivering overdrive pacing therapy to a ventricular region to elicit a sympathetic response.

29. The system of claim 26, wherein:
means for receiving a request to adjust autonomic tone includes means for receiving a signal to increase parasympathetic tone; and
means for delivering an overdrive pacing therapy to at least one cardiac region in response to the request to adjust autonomic tone includes means for delivering overdrive pacing therapy to a ventricular region to elicit a parasympathetic response.
